(19) United States
(12) Patent Application Publication
(10) Pub. No.: US 2004/0111065 A1
(43) Pub. Date: Jun. 10, 2004

(54) Disposable syringe

(76) Inventors: Ming-Jeng Shue, Taichung City (TW); Phillip Shue, Taichung City (TW)

Correspondence Address: Woodard, Emhardt, Naughton,, Moriarty and McNett LLP, Bank One Center/Tower, 111 Monument Circle, Suite 3700, Indianapolis, IN, 46204-5137, US

(21) Appl. No.: 10/388,939

(22) Filed: Mar. 14, 2003

(30) Foreign Application Priority Data
Dec. 10, 2002 (TW) 091135720

(51) Int. Cl. A61M 5/32

(52) U.S. Cl. 604/198

(57) ABSTRACT

A disposable syringe includes an encasing member which is sleevable on a syringe body from a needle hub of the syringe body. A needle cannula is secured to the needle hub and projects outwardly of a surrounding front segment of the encasing member in a position of use. A plunger is slidably disposed in a barrel wall of the syringe body in a longitudinal direction. The syringe body is movable relative to the encasing member in the longitudinal direction between the position of use, where the needle hub is disposed in the surrounding front segment, and a disposal position, where the needle cannula is disposed inwardly of the surrounding front segment as a result of withdrawal of the needle hub from the surrounding front segment.

### BACKGROUND OF THE INVENTION

1. Field of the Invention

This invention relates to a disposable syringe, more particularly to a disposable syringe which enables a needle cannula to be retracted within an encasing member.

2. Description of the Related Art

Exposed needles of conventional disposable syringes may prick the fingers of health care workers after use, and may infect them with contagious fatal diseases, such as AIDS, hepatitis, etc. To reduce the risk of accidents, a disposable syringe with a retractable needle has been developed. However, such a disposable syringe suffers from the following disadvantages in actual use:

1. After injection, a plunger of the disposable syringe has to be first pushed forwardly to couple with the needle and then pulled rearwardly so as to pull the needle into the syringe body. Thereafter, the plunger has to be broken at a certain point to prevent inadvertent pressing of the plunger, which may cause the needle to project from the syringe body once again. Such a retracting procedure is relatively complicated, and considerations have to be given to whether the plunger can be firmly coupled to the needle in order to pull the needle that engages the front end of the syringe body. The coupling of the plunger to the needle and the subsequent pulling of the needle through the plunger are difficult to conduct since the engagement between the needle and the syringe body poses a certain resistance.

2. In order to facilitate retraction of the needle, a greater number of component parts are provided to couple the plunger to the needle, which, however, increases costs and complicates assembly.

3. Due to the provision of more component parts, the amount of residual medication or blood trapped in dead corners of the disposable syringe also increases to result in aggravated environmental pollution problem.

### SUMMARY OF THE INVENTION

The object of the present invention is to provide a disposable syringe which has a relatively simple construction and which enables retraction of a needle after use in a simple operating manner.

According to this invention, the disposable syringe includes a syringe body, a plunger, an encasing member and a retaining member.

The syringe body includes a surrounding barrel wall which has an axis, front and rear surrounding ends opposite to each other in a longitudinal direction parallel to the axis, an outer surrounding wall surface, and an inner surrounding wall surface opposite to the outer surrounding wall surface. The inner surrounding wall surface confines an accommodation chamber. A front end wall extends from the front surrounding end radially and towards the axis to confine an internal port that is in fluid communication with the accommodation chamber. A needle cannula is disposed to extend in the longitudinal direction. A needle hub is disposed to secure the needle cannula to the front end wall such that the needle cannula is in fluid communication with the accommodation chamber through the internal port in the longitudinal direction.

The plunger includes a head portion which is disposed to be movable in the accommodation chamber and which is in slidable contact with the inner surrounding wall surface, and a stem portion which extends from the head portion in the longitudinal direction and outwardly of the rear surrounding end so as to be manually operated.

The encasing member is configured to be sleevable on the syringe body from the needle hub, and includes surrounding front and rear segments. The surrounding front segment surrounds the needle hub, and permits the needle cannula to extend outwardly thereof and to be retractable relative thereto. The surrounding rear segment extends from the surrounding front segment in the longitudinal direction, is to be sleeved on the surrounding barrel wall, and has an inner tubular wall surface which confronts and which is movable relative to the outer surrounding wall surface between a position of use, where the front end wall is closer to the surrounding front segment, and where the needle cannula extends outwardly of the surrounding front segment, and a disposal position, where the front end wall is remote from the surrounding front segment, and where the needle cannula is disposed inwardly of the surrounding front segment as a result of withdrawal of the needle hub from the surrounding front segment.

The retaining member is disposed between the inner tubular wall surface and the outer surrounding wall surface to arrest unforced movement of the surrounding barrel wall in the position of use.

### BRIEF DESCRIPTION OF THE DRAWINGS

Other features and advantages of the present invention will become apparent in the following detailed description of the preferred embodiments of the invention, with reference to the accompanying drawings, in which:

FIG. 1 is an exploded sectional view of the first preferred embodiment of a disposable syringe according to this invention;

FIG. 2 is a schematic view of the first preferred embodiment;

FIG. 3 is a sectional view of the first preferred embodiment in a state of use;

FIG. 4 is a sectional view of the first preferred embodiment in a disposal state;

FIG. 5 is a schematic view of the second preferred embodiment of a disposable syringe according to this invention;

FIG. 6 is a schematic view of the second preferred embodiment in a disposal state;

FIG. 7 is a schematic view of the third preferred embodiment of a disposable syringe according to this invention;

FIG. 8 is a sectional view of the fourth preferred embodiment of a disposable syringe according to this invention; and

FIG. 9 is a sectional view of the fourth preferred embodiment in a disposal state.

### DETAILED DESCRIPTION OF THE PREFERRED EMBODIMENTS

Before the present invention is described in greater detail, it should be noted that same reference numerals have been used to denote like elements throughout the specification.

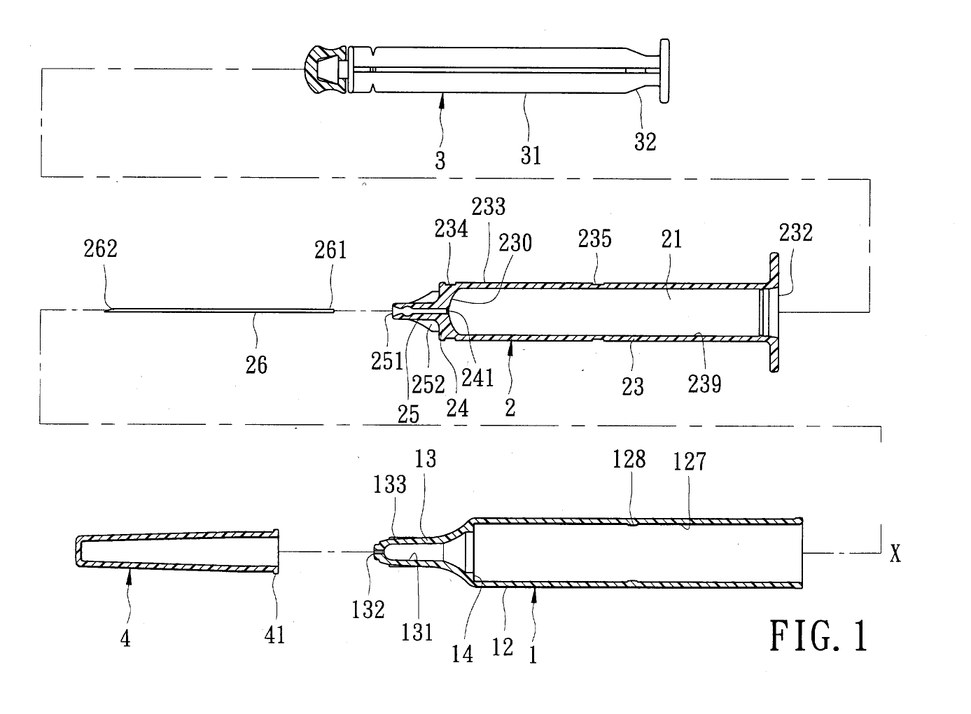

Referring to FIGS. 1 to 3, the first preferred embodiment of a disposable syringe according to the present invention is shown to comprise a syringe body 2, a plunger 3, an encasing member 1, a retaining member, and a tip protector 4.

The syringe body 2 includes a surrounding barrel wall 23, a front end wall 24, a needle cannula 26, and a needle hub 25. The surrounding barrel wall 23 defines an axis (X), and has front and rear surrounding ends 230, 232 opposite to each other in a longitudinal direction parallel to the axis (X). The surrounding barrel wall 23 has an outer surrounding wall surface 233 and an inner surrounding wall surface 239 opposite to the outer surrounding wall surface 233. The inner surrounding wall surface 239 confines an accommodation chamber 21. The front end wall 24 extends from the front surrounding end 230 radially and towards the axis (X) to confine an internal port 241 that is in fluid communication with the accommodation chamber 21. An annular retaining groove 234 is formed in the outer surrounding wall surface 233 adjacent to the front end wall 24.

The needle hub 25 is integrally formed with the front end wall 24, and extends from the front end wall 24 in the longitudinal direction to terminate at an insert end 251. The needle cannula 26 includes a secured end 261 which is secured to the needle hub 25 from the insert end 251, and a cannula body which extends from the secured end 261 in the longitudinal direction to terminate at a tip end 262 that is disposed outwardly of the needle hub 25 such that the needle cannula 26 is in fluid communication with the accommodation chamber 21 through the internal port 241. The needle hub 25 further has a plurality of ribs 252 which are displaced angularly from one another about the axis (X), and each of which extends radially and outwardly.

The plunger 3 includes a head portion 31 which is disposed to be movable in the accommodation chamber 21 and which is in slidable contact with the inner surrounding wall surface 239, and a stem portion 32 which extends from the head portion 31 in the longitudinal direction and outwardly of the rear surrounding end 232 of the surrounding barrel wall 23 so as to be manually operated.

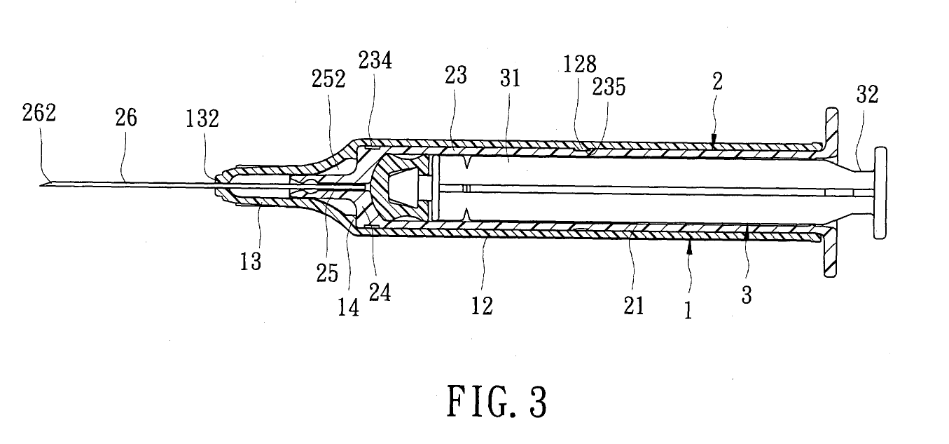

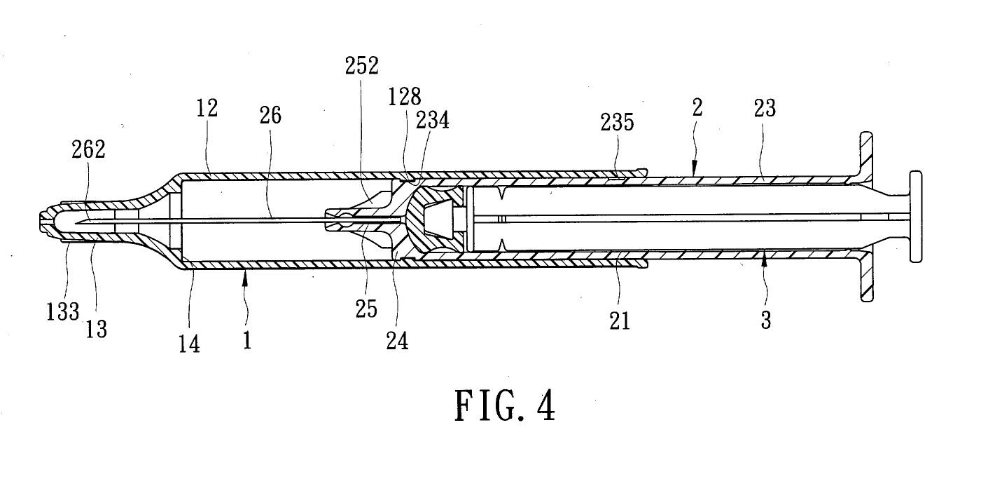

The encasing member 1 is sleevable on the syringe body 2 from the needle hub 25. The encasing member 1 includes a surrounding front segment 13 and a surrounding rear segment 12 which extends from the surrounding front segment 13 in the longitudinal direction. The surrounding front and rear segments 13, 12 have a smaller-diameter inner front wall surface 131 and a larger-diameter inner tubular wall surface 127, respectively, so as to form a surrounding shoulder portion 14 therebetween. The surrounding front segment 13 surrounds the needle hub 25, and has a front open end 132 to permit the needle cannula 26 to extend outwardly thereof and to be retractable relative thereto. The surrounding rear segment 12 is sleeved on the surrounding barrel wall 23. Accordingly, the inner tubular wall surface 127 of the surrounding rear segment 12 confronts and is movable relative to the outer surrounding wall surface 233 of the surrounding barrel wall 23 between a position of use, as shown in FIG. 3, where the front end wall 24 abuts against the surrounding shoulder portion 14, and where the tip end 262 of the needle cannula 26 extends outwardly of the front open end 132 of the front surrounding segment 13, and a disposal position, as shown in FIG. 4, where the front end wall 24 is remote from the surrounding front segment 13, and where the tip end 262 of the needle cannula 26 is disposed inwardly of the surrounding front segment 13 as a result of withdrawal of the needle hub 25 from the surrounding front segment 13. Moreover, when the surrounding front segment 13 is brought to be sleeved on the needle hub 25, the ribs 252 of the needle hub 25 can create an increased friction force at the inner front wall surface 131, thereby establishing greater friction engagement between the needle hub 25 and the surrounding front segment 13.

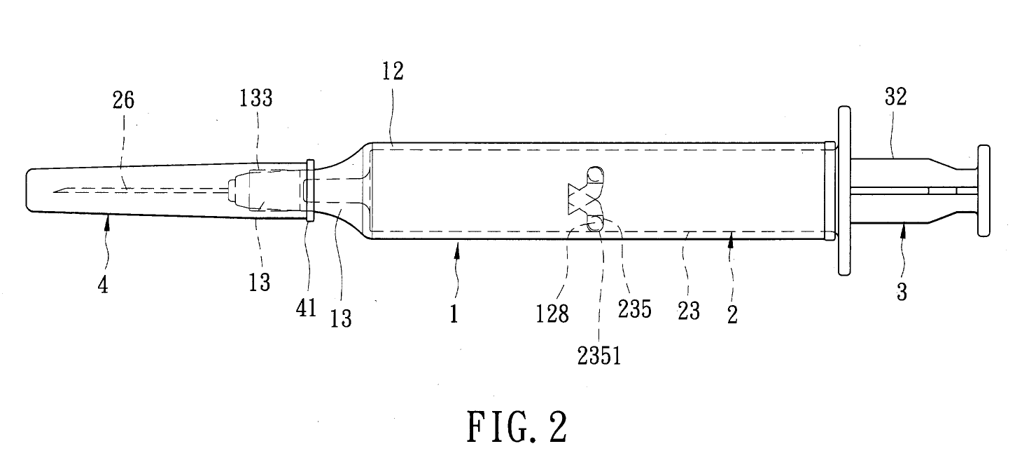

In addition, referring to FIGS. 1 and 2, an outer front wall surface of the surrounding front segment 13 has a rib portion 133 which extends in the longitudinal direction. The tip protector 4 has a sleeve end 41 which is disposed to sleeve on the outer front wall surface of the surrounding front segment 13. The sleeve end 41 includes a groove portion which mates with the rib portion 133 to result in a splined engagement between the tip protector 4 and the outer front wall surface of the surrounding front segment 13, thereby ensuring secure shielding of the needle cannula 26.

The retaining member includes male and female screw thread segments 128, 235 which are disposed on the inner tubular wall surface 127 of the surrounding rear segment 12 and the outer surrounding wall surface 233 of the surrounding barrel wall 23, respectively. The male screw thread segment 128 includes a plurality of angularly displaced ribs and surrounds the axis (X). The female screw thread segment 235 is disposed opposite to the surrounding retaining groove 234 in the longitudinal direction, and includes a plurality of spirally extending grooves (as shown in FIG. 2).

When the encasing member 1 is brought over the needle hub 25 and is sleeved on the syringe body 2, the male screw thread segment 128 is angularly engageable with the female screw thread segment 235 by rotation of the syringe body 2 relative to the encasing member 1 in a counterclockwise direction, until the ribs of the male screw thread segment 128 reach ends 2351 of the grooves of the female screw thread segment 235 so as to restrain the surrounding barrel wall 23 from displacing along the surrounding rear segment 12 in the longitudinal direction, thereby arresting unforced movement of the surrounding barrel wall 23 in the position of use. Therefore, an injection procedure of medication fluid in the accommodation chamber 21 can be performed by pushing the stem portion 32 of the plunger 3 towards the front end wall 24.

When the injection procedure has been completed, referring to FIGS. 2 to 4, the surrounding barrel wall 23 is angularly moved in a clockwise direction relative to the surrounding rear segment 12 so as to disengage the ribs of the male screw thread segment 128 from the grooves of the female screw thread segment 235, and is displaced in the longitudinal direction remote from the surrounding front segment 13. Subsequently, the surrounding barrel wall 23 is pulled in the same direction so as to bring the surrounding barrel wall 23 from the position of use to the disposal position, as shown in FIG. 4, such that the male screw thread segment 128 engages the surrounding retaining groove 234, thereby restraining the surrounding barrel wall 23 from displacing along the surrounding rear segment 12. In this position as described above, the used needle cannula 26 is disposed within the encasing member 1 for safe disposal.

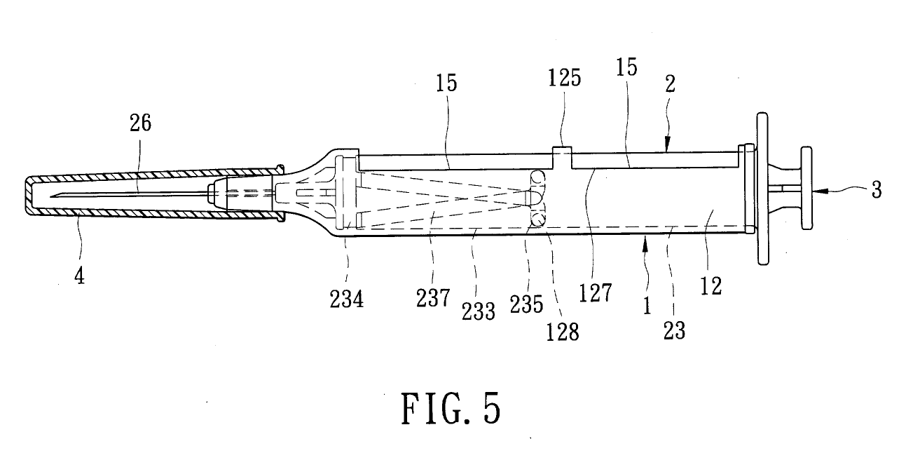

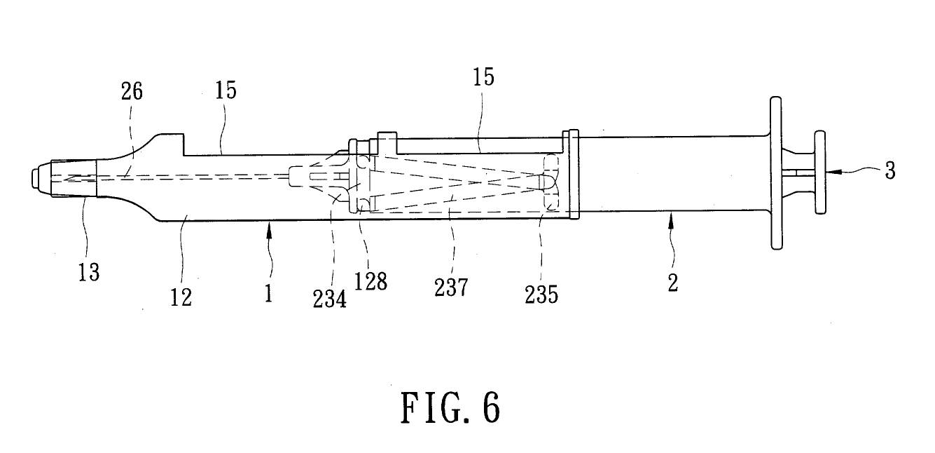

Referring to FIGS. 5 and 6, the second preferred embodiment of the disposable syringe according to this invention is shown to be similar to the aforesaid embodiment in construction. The disposable syringe of this embodiment further includes a guideway 237 which is disposed on the outer surrounding wall surface 233 of the surrounding barrel wall 23, and which spirally extends to interconnect the female screw thread segment 235 and the surrounding retaining groove 234. As such, the male screw thread segment 128 is angularly movable along the guideway 237 between the female screw thread segment 235 and the surrounding retaining groove 234 so as to facilitate the displacement of the surrounding barrel wall 23 from the position of use to the disposal position.

In addition, the surrounding rear segment 12 of the encasing member 1 further has an outer tubular wall surface 125 radially opposite to the inner tubular wall surface 127, and a plurality of slots 15 which are disposed in the outer tubular wall surface 125, which are spaced apart from each other in the longitudinal direction, and which extend through the inner tubular wall surface 127 so as to expose a portion of the surrounding barrel wall 23 of the syringe body 2.

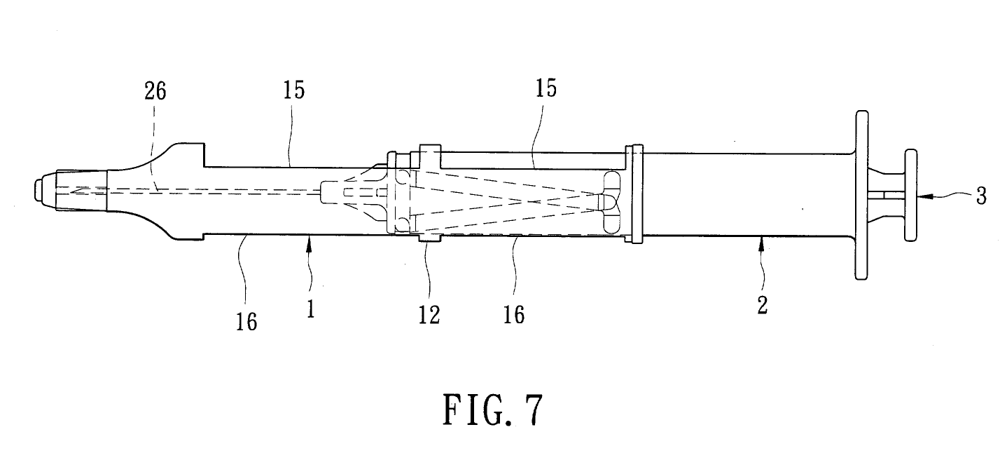

Referring to FIG. 7, the third preferred embodiment of the disposable syringe according to this invention is shown to be similar to the second embodiment in construction. In this embodiment, additional slots 16 are formed through the surrounding rear segment 12 of the encasing member 1, and are disposed diametrically opposite to the slots 15.

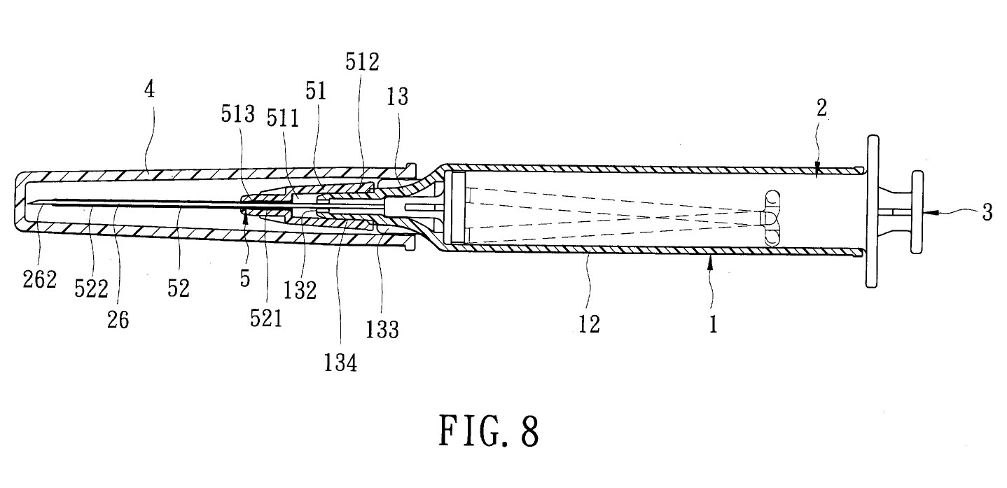

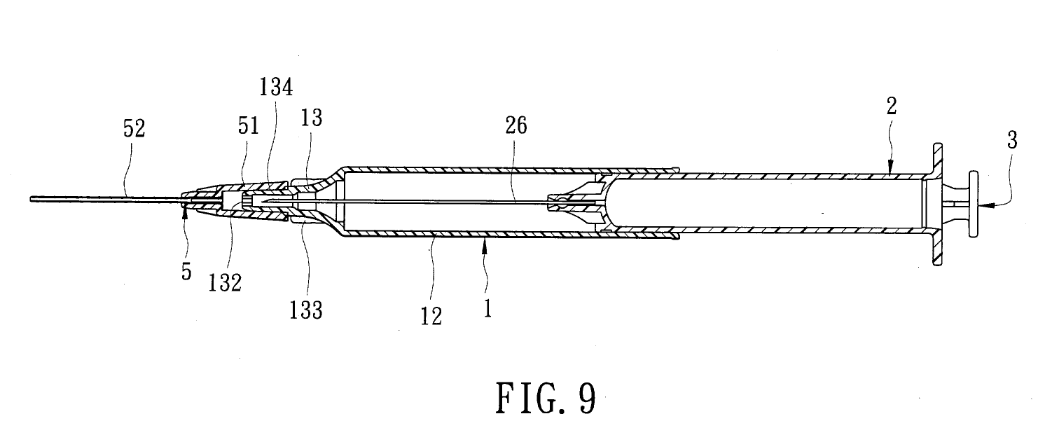

Referring to FIGS. 8 and 9, the fourth preferred embodiment of the disposable syringe according to this invention is shown to be similar to the aforesaid embodiments in construction. The disposable syringe of this embodiment further comprises a catheter device 5 which includes a catheter hub 51 and a tubular catheter 52. In addition, the rib portion 133 of the surrounding front segment 13 is shorter than that of the aforesaid embodiment so that a front surrounding sleeved region 134 is formed on the surrounding front segment 13 and forwardly of the rib portion 133. The catheter hub 51 includes a surrounding hub wall which confines a duct 511 that extends along the axis, and which has a sleeve portion 512 that is sleeved on the front surrounding sleeved region 134, and a tip portion 513 opposite to the sleeve portion 512. The tubular catheter 52 includes a proximate segment 521 which is disposed in the tip portion 513 and which extends along the axis to communicate fluidly with the duct 511, and a distal segment 522 which extends from the proximate segment 521 along the axis to project outwardly of the tip portion 513. The cannula body of the needle cannula 26 passes through the tubular catheter 52 to permit the tip end 262 to project outwardly of the distal segment 522. Hence, after the encasing member 1, in which the syringe body 2 is in the disposal position, has been removed from the catheter hub 51, a transfusion member (not shown) with medication fluid or an empty barrel (not shown) can be connected to the catheter hub 51 for administering the medication fluid into a patient's vein or for drawing blood.

As illustrated, the disposable syringe of this invention has the following advantages:

1. By rotation of the syringe body 2 relative to the encasing member 1 in the clockwise direction to displace the surrounding barrel wall 23 from the position of use to the disposal position, the used needle cannula 26 can be retracted into the encasing member 1, thereby facilitating safe disposal of the disposable syringe.

2. The disposable syringe has a simple construction that is easy to fabricate and assemble at a relatively low cost.

3. Since the plunger 3 is in fit contact with the inner surrounding wall surface 239 of the surrounding barrel wall 23 during the injection procedure, and since there is no component part on the inner surrounding wall surface 239, the amount of residual medication or blood trapped in the accommodation chamber 21 can be minimized.

4. The disposable syringe can serve as an intravenous catheter inserting device for administering medication fluid into a patient's vein or for drawing blood.

While the present invention has been described in connection with what is considered the most practical and preferred embodiments, it is understood that this invention is not limited to the disclosed embodiments but is intended to cover various arrangements included within the spirit and scope of the broadest interpretations and equivalent arrangements.

We claim:

1. A disposable syringe comprising:
a syringe body including
a surrounding barrel wall which has an axis, front and rear surrounding ends opposite to each other in a longitudinal direction parallel to the axis, an outer surrounding wall surface, and an inner surrounding wall surface opposite to said outer surrounding wall surface, said inner surrounding wall surface confining an accommodation chamber,
a front end wall which extends from said front surrounding end radially and towards the axis to confine an internal port in fluid communication with said accommodation chamber,
a needle cannula disposed to extend in the longitudinal direction, and
a needle hub disposed to secure said needle cannula to said front end wall such that said needle cannula is in fluid communication with said accommodation chamber through said internal port in the longitudinal direction;
a plunger including a head portion disposed to be movable in said accommodation chamber and in slidable contact with said inner surrounding wall surface, and a stem portion extending from said head portion in the longitudinal direction and outwardly of said rear surrounding end so as to be manually operated;
an encasing member which is configured to be sleevable on said syringe body from said needle hub, and which includes
a surrounding front segment that surrounds said needle hub, and that permits said needle cannula to extend outwardly thereof and to be retractable relative thereto, and
a surrounding rear segment extending from said surrounding front segment in the longitudinal direction, to be sleeved on said surrounding barrel wall, and having an inner tubular wall surface, said inner tubular wall surface confronting and being movable relative to said outer surrounding wall surface between a position of use, where said front end wall is closer to said surrounding front segment, and where said needle cannula extends outwardly of said surrounding front segment, and a disposal position, where said front end wall is remote from said surrounding front segment, and where said needle cannula is disposed inwardly of said surrounding front segment as a result of withdrawal of said needle hub from said surrounding front segment; and
a retaining member disposed between said inner tubular wall surface and said outer surrounding wall surface to arrest unforced movement of said surrounding barrel wall in the position of use.

2. The disposable syringe of claim 1, wherein said retaining member includes male and female screw thread segments which are disposed on said inner tubular wall surface and said outer surrounding wall surface respectively, and which are angularly engageable once said encasing member is brought over said needle hub and is sleeved on said syringe body so as to restrain said surrounding barrel wall from displacing along said surrounding rear segment in the longitudinal direction, thereby placing said surrounding barrel wall in the position of use.

3. The disposable syringe of claim 2, wherein said male and female screw thread segments are configured such that said surrounding barrel wall is angularly movable in a clockwise direction relative to said surrounding rear segment so as to displace said surrounding barrel wall from the position of use to the disposal position.

4. The disposable syringe of claim 3, further comprising a surrounding retaining groove which is formed in said outer surrounding wall surface adjacent to said front end wall and which is opposite to said female screw thread segment in the longitudinal direction so as to engage said male screw thread segment when said surrounding barrel wall is in the disposal position, thereby restraining said surrounding barrel wall from displacing along said surrounding rear segment.

5. The disposable syringe of claim 4, further comprising a guideway which is disposed on said outer surrounding wall surface, and which extends to interconnect said female screw thread segment and said surrounding retaining groove so as to facilitate the displacement of said surrounding barrel wall from the position of use to the disposal position.

6. The disposable syringe of claim 1, wherein said needle hub is integrally formed with said front end wall and extends from said front end wall in the longitudinal direction.

7. The disposable syringe of claim 6, wherein said surrounding front segment has an inner front wall surface with a diameter which is smaller than that of said inner tubular wall surface so as to form a surrounding shoulder portion therebetween, such that said front end wall abuts against said surrounding shoulder portion in the position of use.

8. The disposable syringe of claim 7, wherein said needle hub has a plurality of ribs displaced angularly from one another about the axis, each of said ribs extending radially and outwardly so as to create an increased friction force at said inner front wall surface, thereby establishing greater friction engagement between said surrounding front segment and said needle hub when said surrounding front segment is sleeved on said needle hub.

9. The disposable syringe of claim 1, wherein said surrounding rear segment further has an outer tubular wall surface radially opposite to said inner tubular wall surface, and a plurality of slots disposed in said outer tubular wall surface and extending through said inner tubular wall surface so as to expose a portion of said surrounding barrel wall.

10. The disposable syringe of claim 1, wherein said surrounding front segment has an outer front wall surface which has a rib portion extending in the longitudinal direction, said disposable syringe further comprising a tip protector which has a sleeve end disposed to sleeve on said outer front wall surface, said sleeve end including a groove portion which mates with said rib portion to result in a splined engagement between said tip protector and said outer front wall surface, thereby ensuring secure shielding of said needle cannula.

11. An intravenous catheter inserting device, comprising:
a syringe body including
a surrounding barrel wall which has an axis, front and rear surrounding ends opposite to each other in a longitudinal direction parallel to the axis, an outer surrounding wall surface, and an inner surrounding wall surface opposite to said outer surrounding wall surface, said inner surrounding wall surface confining an accommodation chamber,
a front end wall which extends from said front surrounding end radially and towards the axis to confine an internal port in fluid communication with said accommodation chamber,
a needle cannula including a secured end and a cannula body which extends from said secured end in the longitudinal direction, and
a needle hub disposed to secure said secured end of said needle cannula to said front end wall such that said needle cannula is in fluid communication with said accommodation chamber through said internal port in the longitudinal direction;
a plunger including a head portion disposed to be movable in said accommodation chamber and in slidable contact with said inner surrounding wall surface, and a stem portion extending from said head portion in the longitudinal direction and outwardly of said rear surrounding end so as to be manually operated;
an encasing member which is configured to be sleevable on said syringe body from said needle hub, and which includes
a surrounding front segment that surrounds said needle hub, and that permits said needle cannula to extend outwardly thereof and to be retractable relative thereto, and
a surrounding rear segment extending from said surrounding front segment in the longitudinal direction, to be sleeved on said surrounding barrel wall, and having an inner tubular wall surface, said inner tubular wall surface confronting and being movable relative to said outer surrounding wall surface between a position of use, where said front end wall is closer to said surrounding front segment, and where said needle cannula extends outwardly of said surrounding front segment, and a disposal position, where said front end wall is remote from said surrounding front segment, and where said needle cannula is disposed inwardly of said surrounding front segment as a result of withdrawal of said needle hub from said surrounding front segment;
a retaining member disposed between said inner tubular wall surface and said outer surrounding wall surface to arrest unforced movement of said surrounding barrel wall in the position of use;
a catheter hub including a surrounding hub wall which confines a duct that extends along the axis, and which has a sleeve portion that is sleeved on said surrounding front segment, and a tip portion opposite to said sleeve portion along the axis; and
a tubular catheter including a proximate segment which is disposed in said tip portion and which extends along the axis to communicate fluidly with said duct, and a distal segment which extends from said proximate segment along the axis to project outwardly of said tip portion,
said cannula body of said needle cannula extending along and in said tubular catheter to terminate at a tip end that projects outwardly of said distal segment of said tubular catheter.

12. The intravenous catheter inserting device of claim 11, wherein said surrounding front segment has an outer front wall surface which has a rib portion extending in the longitudinal direction, and a front surrounding sleeved region disposed forwardly of said rib portion such that said sleeve portion of said surrounding hub wall is sleeved on said front surrounding sleeved region, said disposable syringe further comprising a tip protector which has a sleeve end disposed to sleeve on said outer front wall surface so as to shield said needle cannula, said catheter hub and said tubular catheter.
